(19) United States
(12) Patent Application Publication
(10) Pub. No.: US 2025/0389687 A1
(43) Pub. Date: Dec. 25, 2025

(54) METHOD OF FORMING ELECTRICAL SIGNAL SENSOR

(71) Applicant: National Yang Ming Chiao Tung University, Hsinchu City (TW)

(72) Inventors: Hsin-Chieh Lin, Hsinchu City (TW); Yuan-Hao Xiao, Hsinchu City (TW)

(73) Assignee: National Yang Ming Chiao Tung University, Hsinchu City (TW)

(21) Appl. No.: 19/310,202

(22) Filed: Aug. 26, 2025

Related U.S. Application Data
(62) Division of application No. 18/302,390, filed on Apr. 18, 2023.

(30) Foreign Application Priority Data
Jan. 4, 2023 (TW) 112100283

(51) Int. Cl. G01N 27/327

(52) U.S. Cl. CPC G01N 27/3271; G01N 27/3277

(57) ABSTRACT

The present disclosure relates to a method of forming electrical signal sensor. The method includes the following operations. An oxidoreductase and an amphiphilic molecule are dissolved into a solvent to form a solution, in which the amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The solution is coated on an electrode layer. The solution is dried.

### CROSS-REFERENCE TO RELATED APPLICATION

This present application is a divisional application of the U.S. application Ser. No. 18/302,390, filed Apr. 18, 2023, which claims priority to Taiwan Application Serial Number 112100283, filed Jan. 4, 2023, which is herein incorporated by reference in its entirety.

### BACKGROUND

### Field of Invention

The present disclosure relates to an electrical signal sensing composition, an electrical signal sensor, and a method of forming an electrical signal sensor.

### Description of Related Art

Biosensors can sense the electrical signals associated with the electrochemical reactions of the organisms. For example, glucose sensors can sense the amount of glucose in the body fluids to monitor the changes in blood sugar. With the increasing number of diabetics and the related medical expenses, the demand for glucose sensors is also increasing. Therefore, it is necessary to develop a novel biosensor and a method of forming the same. This novel biosensor can not only sense the electrical signal of the bioelectrochemical reactions but also increase the signal strength and improve the detection limit to achieve repetitive and long-term sensing.

### SUMMARY

The present disclosure provides a method of forming electrical signal sensor. The method includes the following operations. An oxidoreductase and an amphiphilic molecule are dissolved into a solvent to form a solution, in which the amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The solution is coated on an electrode layer. The solution is dried.

In some embodiments, the amphiphilic molecule includes sodium n-octyl sulfate, sodium n-decyl sulfate, sodium dodecyl sulfate, sodium n-tetradecyl sulfate, or combinations thereof.

In some embodiments, the oxidoreductase includes glucose oxidase, glucose dehydrogenase, pyruvate oxidase, catalase, xanthine oxidase, acetylcholinesterase, lactate oxidase, urate oxidase, pyrroloquinoline quinine glucose dehydrogenase-A, pyrroloquinoline quinine glucose dehydrogenase-B, NAD(P)-dependent glutamate dehydrogenase, FAD-dependent glutamate dehydrogenase, uricase, cholesterol oxidase, sulfurase, or combinations thereof.

In some embodiments, the oxidoreductase includes an aggregated particle, and a particle size of the aggregated particle is from 10 nm to 5000 nm.

In some embodiments, the particle size of the aggregated particle is from 50 nm to 1000 nm.

In some embodiments, a weight ratio of the oxidoreductase to the amphiphilic molecule is from 1:0.1 to 1:50.

In some embodiments, the weight ratio of the oxidoreductase to the amphiphilic molecule is from 1:0.1 to 1:10.

In some embodiments, the electrode layer includes gold, silver, platinum, aluminum, iridium, titanium, steel, stainless steel, gold alloy, platinum alloy, aluminum alloy, iridium alloy, titanium alloy, or combinations thereof.

In some embodiments, the oxidoreductase and the amphiphilic molecule are dissolved into the solvent at to form the solution a temperature range from 20° C. to 30° C.

In some embodiments, the operation of coating the solution on the electrode layer is performed by dropping the solution onto a whole upper surface of the electrode layer.

The present disclosure also provides another method of forming an electrical signal sensor. The method includes the following operations. An electron transfer layer is disposed on an electrode layer. A solution is coated on the electron transfer layer, in which the solution includes an oxidoreductase, an amphiphilic molecule and a solvent, in which the amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The solution is dried.

In some embodiments, the amphiphilic molecule includes sodium n-octyl sulfate, sodium n-decyl sulfate, sodium dodecyl sulfate, sodium n-tetradecyl sulfate, or combinations thereof.

In some embodiments, the oxidoreductase includes glucose oxidase, glucose dehydrogenase, pyruvate oxidase, catalase, xanthine oxidase, acetylcholinesterase, lactate oxidase, urate oxidase, pyrroloquinoline quinine glucose dehydrogenase-A, pyrroloquinoline quinine glucose dehydrogenase-B, NAD(P)-dependent glutamate dehydrogenase, FAD-dependent glutamate dehydrogenase, uricase, cholesterol oxidase, sulfurase, or combinations thereof.

In some embodiments, the electron transfer layer includes a conductive carbon material, a conductive polymer, or a combination thereof.

In some embodiments, the conductive carbon material comprises graphite, graphene, single-wall carbon nanotube, multi-wall carbon nanotube, or combinations thereof, and the conductive polymer comprises polypyrrole, polyaniline, polythiophene, poly(3,4-ethylenedioxythiophene), poly-p-styrene, polyacetylene, polyphenylacetylene, polyphenylene sulfide, polybenzene, polythiazole, or combinations thereof.

### BRIEF DESCRIPTION OF THE DRAWINGS

When reading the accompanying figures of the present disclosure, it is recommended to understand the various aspects of the present disclosure from the following description. It is noted that according to standard industry practice, sizes of various features may not be drawn in scale. For a clear discussion, the sizes of various features may be arbitrarily increased or decreased. In addition, to simplify the illustration, the accustomed structures and components may be drawn schematically in the figures.

FIG. 1 is a cross-sectional schematic of an electrical signal sensor including an electrical signal sensing composition according to some embodiments of the present disclosure.

FIG. 2 is a cross-sectional schematic of an electrical signal sensor including an electrical signal sensing composition according to other embodiments of the present disclosure.

FIG. 3 is a flow chart of a method of forming an electrical signal sensor including an electrical signal sensing composition according to some embodiments of the present disclosure.

FIG. 4 is a diagram of current changing with time for an electrical signal sensor using an electrical signal sensing composition according to some embodiments of the present disclosure.

FIG. 5 is a diagram of current changing with time for an electrical signal sensor using an electrical signal sensing composition according to other embodiments of the present disclosure.

FIG. 6 is a diagram of relative current density changing with time for an electrical signal sensor using an electrical signal sensing composition according to some embodiments of the present disclosure.

### DETAILED DESCRIPTION

To make the description of the present disclosure more detailed and complete, the following provides an illustrative description of the aspects of the embodiments and the specific embodiments. This does not limit the implementation of the present disclosure to only one form. The embodiments of the present disclosure may be combined or substituted in beneficial instances, and some other embodiments may be added without further statement or explanation.

In addition, spatially relative terms, such as below and above, describe the relationship of one component or feature to another component or feature in the figures of the present disclosure. In addition to the orientation depicted in the figures, spatially relative terms intend to cover the different orientations of the device in use or operation. For example, the device may be oriented otherwise (e.g., rotating 90 degrees or in other directions), and the spatially relative terms of the present disclosure may accordingly interpret. In the present disclosure, unless otherwise stated, the same reference numbers of the components in the different figures represent the same or similar components formed in the same or similar methods by the same or similar materials.

The present disclosure relates to an electrical signal sensing composition. The electrical signal sensing composition includes an oxidoreductase and an amphiphilic molecule. The amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The oxidoreductase of the present disclosure can catalyze the biologically relevant tested sample to undergo a redox reaction. The amphiphilic molecule acts as a medium to improve the electron transfer in the redox reaction, thereby enhancing the conduction effect of the electrical signal sensing composition. The electrical signal sensor with the electrical signal sensing composition of the present disclosure can improve the performance, such as increased electrical signal, smaller detection limit, increases detection range, better detection sensitivity, etc. Next, the electrical signal sensing composition of the present disclosure is described in detail according to the embodiments.

The oxidoreductase catalyzes the biologically relevant tested sample to undergo a redox reaction. In some embodiments, the oxidoreductase includes but is not limited to glucose oxidase, glucose dehydrogenase, pyruvate oxidase, catalase, xanthine oxidase, acetylcholinesterase, lactate oxidase, urate oxidase, pyrroloquinoline quinine glucose dehydrogenase-A (PQQGDH-A), pyrroloquinoline quinine glucose dehydrogenase-B (PQQGDH-B), NAD(P)-dependent glutamate dehydrogenase (NAD(P)-GDH), FAD-dependent glutamate dehydrogenase, uricase (FADGDH), cholesterol oxidase, sulfurase, or combinations thereof. As long as the oxidoreductase can catalyze the tested sample to undergo a redox reaction, it is the oxidoreductase the present disclosure intends to cover. In the embodiments that the oxidoreductase includes the glucose oxidase, the tested sample may be glucose in the body fluids associated with the living organism, in which the glucose oxidase catalyzes the glucose to perform a redox reaction. In some embodiments, the oxidoreductase includes an aggregated particle, such as a nanoparticle, a microparticle, or a combination thereof, which is aggregated by the oxidoreductase. It is noted that the oxidoreductase of the present disclosure intends to cover the oxidoreductase that aggregates to be the aggregated particle and that is not aggregated to be the aggregated particle. In some embodiments, a particle size of the aggregated particle is from 10 nm to 5000 nm, for example, 10 nm, 50 nm, 100 nm, 500 nm, 700 nm, 1000 nm, 2000 nm, 3000 nm, or 5000 nm, in which 50 nm to 1000 nm is preferable. Compared with the oxidoreductase that does not aggregate to be the aggregated particle, the oxidoreductase that aggregates to be the aggregated particle further makes the electrical signal sensing composition improve the performance of the electrical signal sensor, such as increased electrical signal, smaller detection limit, increased detection range, better detection sensitivity, etc. In some embodiments, the surface of the oxidoreductase is modified or not modified. The surface of the oxidoreductase being modified can prevent biological attachment, which makes the electrical signal sensing composition of the present disclosure can apply to the implanted or invasive electrical signal sensor. In some embodiments, the surface of the oxidoreductase being modified preferably includes an amine group, thiol, or a combination thereof.

The amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The amphiphilic molecule improves the electron transfer in the redox reaction catalyzed by the oxidoreductase. The amphiphilic molecule includes a hydrophilic end (e.g., a sulfuric acid end, a sulfate end, a sulfonic acid end, a sulfonate end, an ammonium end, an ammonium salt end, a phosphoric acid end, a phosphate end, a carboxylic acid end, a carboxylate end, a boric acid end, a borate end) and a hydrophobic end (e.g., a alkyl terminal end away from the above-mentioned hydrophilic ends); therefore, the amphiphilic molecule, the oxidoreductase, and the organism-related body fluid including the tested sample are mixed well, which helps the electron transfer in the electrical signal sensing composition. The hydrophilic end of the amphiphilic molecule can have charges on it after dissociation, thereby enhancing the conductivity of the electrical signal sensing compositions. The amphiphilic molecule includes a carbon backbone having a number of carbons, in which the number is any integer in a range from 5 to 20, to improve the hydrophobicity at the hydrophobic end of the amphiphilic molecule. In some embodiments, the amphiphilic molecule is a solid but not an ionic liquid at room temperature (a temperature range from 20° C. to 30° C., e.g., 25° C.) and is soluble in an appropriate solvent, so it is easier to implement and save cost compared with forming an ionic liquid of the amphiphilic molecule and performing the subsequent treatment hereafter (see below for details of a method of forming an electrical signal sensor). In some embodiments, the preferable amphiphilic molecule includes but is not limited to sodium n-octyl sulfate (SOS), sodium n-decyl sulfate (SnDS), sodium dodecyl sulfate (SDS), sodium n-tetradecyl sulfate (STS), or combinations thereof. As long as the amphiphilic molecule includes the hydrophilic and hydrophobic ends to mix well with the oxidoreductase and the tested sample to improve the conductivity as mentioned above and is a solid, not an ionic liquid, and is soluble in an appropriate solvent at room temperature, it is the amphiphilic molecule the present disclosure intends to cover.

In some embodiments, a weight ratio of the oxidoreductase to the amphiphilic molecule is 1:0.1 to 1:50, for example, 1:0.1, 1:0.9, 1:5, 1:8, 1:10, 1:20, 1:30, 1:40, or 1:50, in which 1:0.1 to 1:10 is preferable. When the weight ratio is not within the above-recommended range, for example, the amphiphilic molecule being too little or the oxidoreductase being too much, the electron transfer in the electrical signal sensing composition may be poor, so the electrical signal decreases. Or for example, when the amphiphilic molecule is too much, the electrical signal sensing composition may detach from the electrode layer or the electron transfer layer discussed below to make the function of sensing fail. Or for example, when the oxidoreductase is too much, the signal of the electrical signal sensor may saturate.

The present disclosure also relates to an electrical signal sensor including the above-mentioned electrical signal sensing composition. The electrical signal sensor includes an electrode layer and a sensing layer. The sensing layer is located on the electrode layer, in which the sensing layer includes the electrical signal sensing composition of any one of the above-mentioned embodiments. The electrical signal sensor of the present disclosure improves the performance by including the electrical signal sensing composition, such as increased electrical signal, smaller detection limit, increased detection range, better detection sensitivity, etc. Next, the electrical signal sensor of the present disclosure is described in detail according to the embodiments.

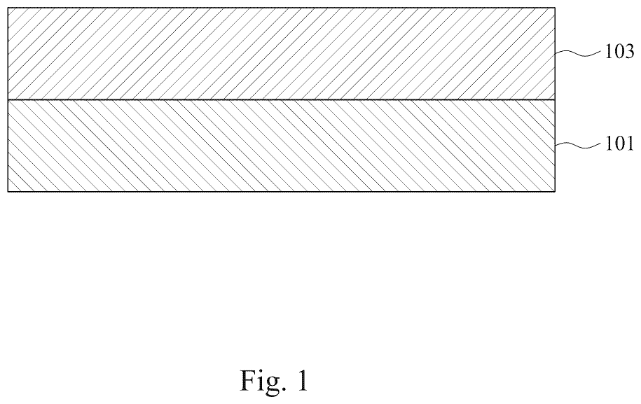

FIG. 1 is a cross-sectional schematic of the electrical signal sensor including the electrical signal sensing composition according to some embodiments of the present disclosure. The electrical signal sensor includes the electrode layer 101 and the sensing layer 103. The electrode layer 101 and the sensing layer 103 of FIG. 1 are described in detail below.

The electrode layer 101 transmits the electrical signal received from the sensing layer 103 located thereon to an external circuit (not drawn), and therefore a quantized electrical signal is obtained by measuring the current. In some embodiments, the electrode layer 101 includes but is not limited to gold, silver, platinum, aluminum, iridium, titanium, steel, stainless steel, gold alloy, platinum alloy, aluminum alloy, iridium alloy, titanium alloy, or combinations thereof. As long as the electrode layer has good conductivity and does not affect the electron transfer in the sensing layer 103, it is the electrode layer 101 the present disclosure intends to cover.

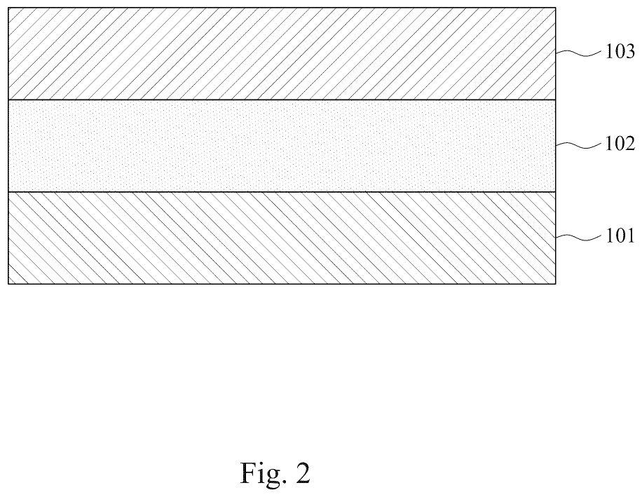

The sensing layer 103 includes the electrical signal sensing composition of any one of the embodiments described above. Details of the electrical signal sensing composition can refer to the context above, and they will not be repeated hereafter. Detecting the content of the tested sample in the body fluid can be performed by dropping the body fluid including the tested sample on the sensing layer 103, or directly immersing the sensing layer 103 into the body fluid including the tested sample. The principle of the oxidoreductase and the amphiphilic molecular to sense the tested sample in the electrical signal sensing composition of the sensing layer 103 can refer to the context above, and the detail will not be repeated hereafter. In some embodiments, the sensing layer 103 is a single layer as shown in FIG. 1 (or FIG. 2 discussed below), but it actually can include more than one layer. As long as the sensing layer includes the electrical signal sensing composition, it is the sensing layer 103 the present disclosure intends to cover.

FIG. 2 is a cross-sectional schematic of the electrical signal sensor including the electrical signal sensing composition according to other embodiments of the present disclosure. The difference between FIG. 2 and FIG. 1 is that the electrical signal sensor of FIG. 2 further includes an electron transfer layer 102 located between the electrode layer 101 and the sensing layer 103. The electrode layer 101 and the sensing layer 103 shown in FIG. 2 are substantially the same as the electrode layer 101 and the sensing layer 103 shown in FIG. 1, so their details will not be repeated hereafter. The electron transfer layer 102 of FIG. 2 will be described in detail below.

The electron transfer layer 102 helps the electrical signal generated in the sensing layer 103 to be more easily transmitted to the electrode layer 101, thereby enhancing the electrical signal detected by the electrical signal sensor. In some embodiments, the preferable material of the electron transfer layer 102 includes but is not limited to a conductive carbon material, a conductive polymer, or a combination thereof. As long as the electron transfer layer has good conductivity to improve the conductivity between the electrode layer 101 and the sensing layer 103, it is the electron transfer layer 102 the present disclosure intends to cover. In some embodiments, the preferable conductive carbon material includes graphite, graphene, single-wall carbon nanotube (SWCNT), multi-wall carbon nanotube (MWCNT), or combinations thereof. In some embodiments, the preferable conductive polymer includes polypyrrole, polyaniline, polythiophene, poly(3,4-ethylenedioxythiophene), poly-p-styrene, polyacetylene, polyphenylacetylene, polyphenylene sulfide, polybenzene, polythiazole, or combinations thereof, in which a more preferable conductive polymer includes a combination of poly(3,4-ethylenedioxythiophene) and poly-p-styrene, and polypyrrole. In some embodiments, the electron transfer layer 102 further includes polystyrene sulfonate to enhance the solubility of the above-mentioned conductive material.

The present disclosure yet also relates to a method of forming the above-mentioned electrical signal sensor. Details of the electrical signal sensor and the electrical signal sensing composition included therein can refer to the context above, so they are not repeated below. The method of the present disclosure includes the following operations. The oxidoreductase and the amphiphilic molecule are dissolved into a solvent to form a solution, in which the amphiphilic molecule includes alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof. The solution is coated on the electrode layer. The solution is dried. The method of the present disclosure uses the amphiphilic molecule that is solid, not an ionic liquid and is soluble in an appropriate solvent, so it is easier to implement and saves cost compared with forming an ionic liquid of the amphiphilic molecule and performing the subsequent operations. The electrical signal sensor formed by using the method of the present disclosure includes the electrical signal sensing composition, so the performance enhances, such as increased electrical signal, smaller detection limit, increased detection range, better detection sensitivity, etc. Next, the method of the present disclosure is described in detail according to the embodiments.

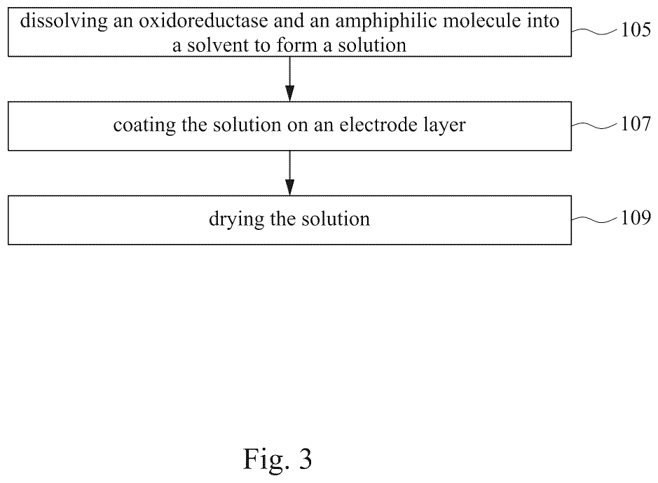

FIG. 3 is a flowchart of the method of forming the electrical signal sensor including the electrical signal sensing composition according to some embodiments of the present disclosure. The method includes an operation 105, an operation 107, and an operation 109. The operation 105, the operation 107, and the operation 109 of FIG. 3 are described in detail below.

In the operation 105, the oxidoreductase and the amphiphilic molecule are dissolved into a solvent to form a solution. As above-mentioned, the amphiphilic molecule is a solid, not an ionic liquid, and is soluble in an appropriate solvent at room temperature, so the amphiphilic molecule and the oxidoreductase can be mixed thoroughly for use in the subsequent operation merely by dissolving them into an appropriate solvent at room temperature. Compared with other processes (e.g., heating processes) forming an ionic liquid of the amphiphilic molecule and using this ionic liquid to mix with the oxidoreductase, the present disclosure is easier to implement and saves cost. In some embodiments, the appropriate solvent includes deionized water, pentylene glycol, or a combination thereof. In some embodiments, a volume ratio of the pentylene glycol to the deionized water is 1:8˜12, for example, 1:10.

In the operation 107, the solution formed in the operation 105 is coated on the electrode layer. In some embodiments, the solution is coated on the electrode layer 101 shown in FIG. 1. In some embodiments, the solution is coated on the electron transfer layer 102 on the electrode layer 101 shown in FIG. 2. In some embodiments, the coating is performed by dropping the solution onto the whole upper surface of the electrode layer 101 or the electron transfer layer 102.

In the operation 109, the solution is dried to volatilize the solvent to form the sensing layer 103 shown in FIG. 1 or FIG. 2. In some embodiments, the operation 107 and the operation 109 may be performed repeatedly, so there may be more than one sensing layer 103 on the electrode layer 101 or the electron transfer layer 102.

The electrical signal sensing composition, the electrical signal sensor including the electrical signal sensing composition, and the method of forming the electrical signal sensor of the present disclosure have the following advantages. The electrical signal sensing composition has good electron transfer, which can improve the performance of the electrical signal sensor, such as increased electrical signal, smaller detection limit, increased detection range, better detection sensitivity, etc. Therefore, the electrical signal sensor can still detect a discernible electrical signal after a long period and continuous use. Moreover, the electrical signal sensor is used in a wide range, for example, sensing the tested sample in the body fluids including blood, digestive fluids, saliva, tears, interstitial fluids, sweat, urine, or combinations thereof. Moreover, the electrical signal sensor can quickly determine the content of the tested sample in the body fluid. In addition, the electrical signal sensor formed in the present disclosure costs low but has high market demand, and therefore there are a lot of business opportunities. For example, there are more than 460 million people with diabetes worldwide, and annual medical expense costs at least 727 billion US dollars. Therefore, the electrical signal sensor of the present disclosure that can sense glucose will earn a big commercial success because of its low cost and high market demand. In addition, the electrical signal sensor of the present disclosure can sense glucose in the blood daily, and the electrical signal almost does not attenuate after two weeks and the electrical signal attenuates less than 20% after forty days. In addition, the electrical signal sensor of the present disclosure can sense a wide range of glucose concentrations in the blood, for example, 3.3 mg/dL to 720 mg/dL (preferable being 5 mg/dL to 500 mg/dL), and a low glucose concentration in the blood, for example, detection limits less than 5 mg/dL (smallest to 3.3 mg/dL).

Next, only some embodiments (Embodiment 1 to Embodiment 8) are provided to explain the electrical signal sensor of the present disclosure. It is noted that these embodiments are used to understand the present disclosure better and are not intended to limit the scope of the present disclosure. Even without a further explanation, the electrical signal sensors of the embodiments shown above also perform well.

In Embodiment 1, the electrode layer includes platinum, the electron transfer layer includes poly(3,4-ethylenedioxythiophene) and polystyrene sulfonate, the oxidoreductase of the electrical signal sensing composition in the sensing layer includes glucose oxidase and does not include the aggregated particle, and the amphiphilic molecule of the electrical signal sensing composition in the sensing layer includes sodium dodecyl sulfate, in which the weight ratio of the oxidoreductase to the amphiphilic molecule is 1:1.15.

In Comparative Embodiment 1, the electrode layer includes platinum, the electron transfer layer includes poly(3,4-ethylenedioxythiophene) and polystyrene sulfonate, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 2, the electrode layer includes platinum, the electron transfer layer includes polypyrrole, the oxidoreductase of the electrical signal sensing composition in the sensing layer includes glucose oxidase and does not include the aggregated particle, and the amphiphilic molecule of the electrical signal sensing composition in the sensing layer includes sodium dodecyl sulfate, in which the weight ratio of the oxidoreductase to the amphiphilic molecule is 1:1.15.

In Comparative Embodiment 2, the electrode layer includes platinum, the electron transfer layer includes polypyrrole, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 3, the electrode layer includes platinum, the electron transfer layer includes the single-wall carbon nanotube, the oxidoreductase of the electrical signal sensing composition in the sensing layer includes glucose oxidase and does not include the aggregated particle, and the amphiphilic molecules of the electrical signal sensing composition in the sensing layer include sodium dodecyl sulfate, in which the weight ratio of the oxidoreductase to the amphiphilic molecules 1:1.15.

In Comparative Embodiment 3, the electrode layer includes platinum, the electron transfer layer includes a single-wall carbon nanotube, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 4, the electrode layer includes platinum, the electron transfer layer includes the multi-wall carbon nanotube, the oxidoreductase of the electrical signal sensing composition in the sensing layer includes glucose oxidase and does not include the aggregated particle, and the amphiphilic molecule of the electrical signal sensing composition in the sensing layer includes sodium dodecyl sulfate, in which the weight ratio of the oxidoreductase to the amphiphilic molecule is 1:1.15.

In Comparative Embodiment 4, the electrode layer includes platinum, the electron transfer layer includes the multi-wall carbon nanotube, the oxidoreductase of the electrical signal sensing composition in the sensing layer includes glucose oxidase and does not include the aggregated particles, and the electrical signal sensing composition in the sensing layer does not include the amphiphilic molecule.

In Embodiment 5, the electrode layer includes platinum, and the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the amphiphilic molecule of the electrical signal sensing composition in the sensing layer includes sodium n-octyl sulfate, in which the weight ratio of the oxidoreductase to the amphiphilic molecule is 1:0.9.

In Comparative Embodiment 5, the electrode layer includes platinum, the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 6, the electrode layer includes platinum, the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the amphiphilic molecules of the electrical signal sensing composition in the sensing layer includes sodium n-decyl sulfate, in which the weight ratio of the oxidoreductase to the amphiphilic molecule is 1:1.04.

In Comparative Embodiment 6, the electrode layer includes platinum, the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 7, the electrode layer includes platinum, the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the amphiphilic molecule of the electrical signal sensing composition in the sensing layer includes sodium n-tetradecyl sulfate, in which the weight ratio of the oxidoreductase to the amphiphilic molecule is 1:1.27.

In Comparative Embodiment 7, the electrode layer includes platinum, the electron transfer layer includes the combination of poly(3,4-ethylenedioxythiophene) and poly-p-styrene, the oxidoreductase of the electrical signal sensing composition in the sensing layer includes glucose oxidase and does not include the aggregated particle, and the electrical signal sensing composition in the sensing layer does not include the amphiphilic molecule.

In Embodiment 8, the electrode layer includes platinum, the electron transfer layer includes poly(3,4-ethylenedioxythiophene), the oxidoreductase of the electrical signal sensing composition in the sensing layer includes glucose oxidase and includes the aggregated particle (particle size being 150 nm), and the amphiphilic molecule of the electrical signal sensing composition in the sensing layer includes sodium dodecyl sulfate, in which the weight ratio of the oxidoreductase to the amphiphilic molecule is 1:7.2.

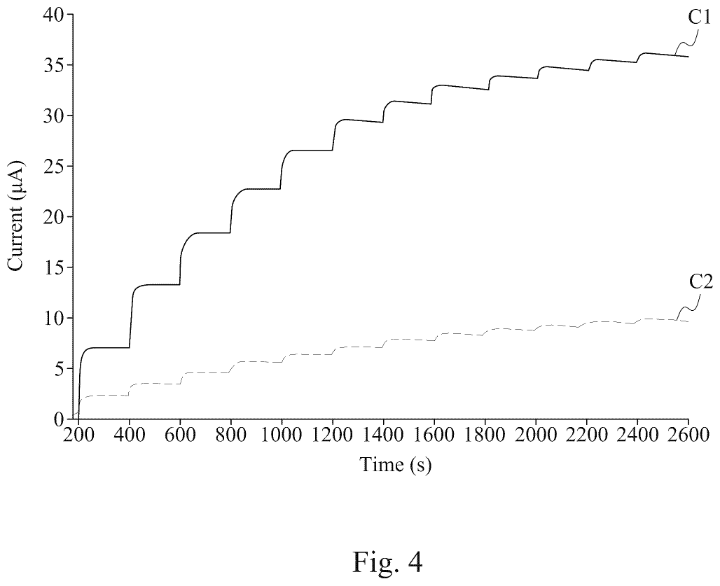

Comparing Embodiment 1 to Embodiment 7 that includes the amphiphilic molecule with Comparative Embodiment 1 to Comparative Embodiment 7 that does not include the amphiphilic molecule, when the electrical signal sensor includes the amphiphilic molecule, the electrical signal strength enhances significantly. Taking Embodiment 1 and Comparative Embodiment 1 as an example, the comparison of the two is shown in FIG. 4. FIG. 4 is a diagram of the current in the electrical signal sensor changing with time. In FIG. 4, curve C1 corresponds to Embodiment 1, and curve C2 corresponds to Comparative Embodiment 1. In FIG. 4, glucose is added to the sensing layer as time increases, and the current signal increases stepwise, in which each step corresponds to an addition of 36 mg/dl glucose. As shown in FIG. 4, the current signal of curve C1 of Embodiment 1 is significantly larger than the current signal of curve C2 of Comparative Embodiment 1. The comparisons of the current changing with time for Embodiment 2 to Embodiment 7 with Comparative Embodiment 2 to Comparative Embodiment 7 are respectively the same as FIG. 4, i.e., the current signals of Embodiment 2 to Embodiment 7 are significantly larger than the current signals of Comparative Embodiment 2 to Comparative Embodiment 7, although the respective figures are not provided in the present disclosure.

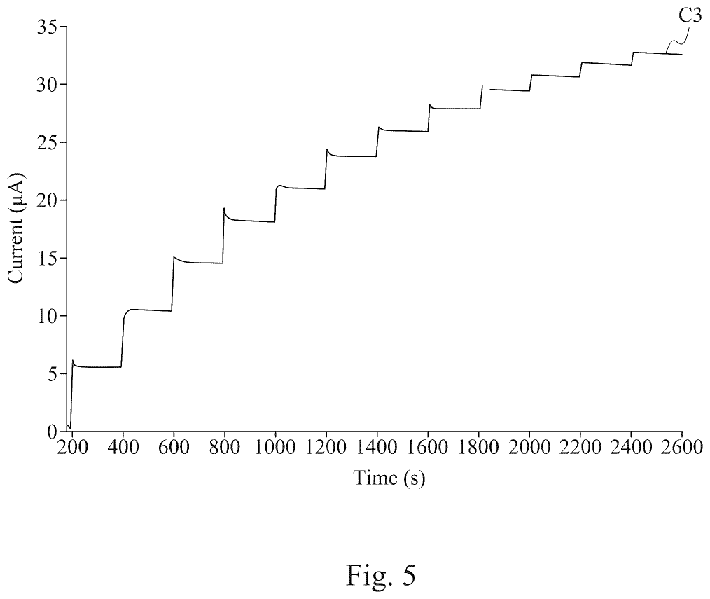

Embodiment 8 including the oxidoreductase with the aggregated particle is compared with Comparative Embodiment 1 including the oxidoreductase without the aggregated particle. FIG. 5 is a diagram of the current in the electrical signal sensor changing with time. In FIG. 5, curve C3 corresponds to Embodiment 8. In FIG. 5, glucose is added to the sensing layer as time increases, and the current signal increases stepwise, in which each step corresponds to an addition of 36 mg/dl glucose. As shown in FIG. 5, although the glucose oxidase in Embodiment 8 (the weight ratio of the oxidoreductase to the amphiphilic molecule being 1:7.2) is significantly smaller than Embodiment 1 (the weight ratio of the oxidoreductase to the amphiphilic molecule being 1:1.15), the current signal of curve C3 of Embodiment 8 that includes the aggregated particle is still strong and comparable to the current signal of curve C1 of Embodiment 1 (see FIG. 4). The oxidoreductase that includes the aggregated particle significantly improves the efficiency of the electrical signal sensor, and the amount of the oxidoreductase that includes the aggregated particle does not need to be too much to achieve a similar effect of the oxidoreductase that does not include the aggregated particle.

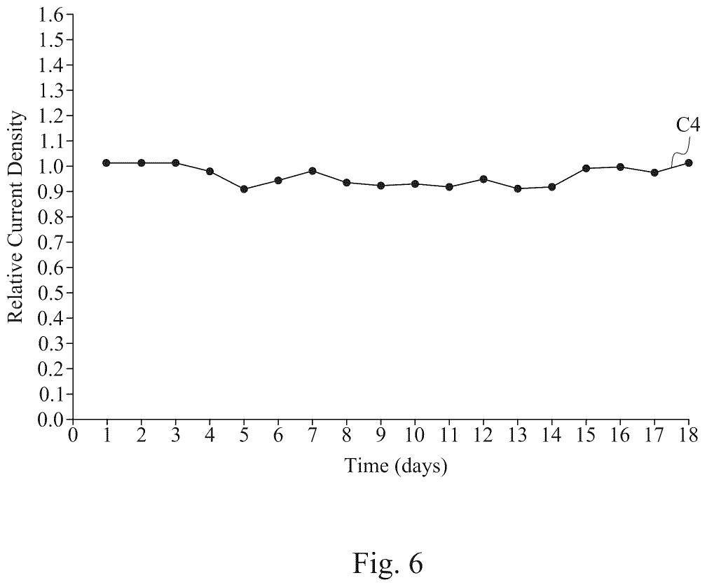

The above-mentioned specific embodiments show that the electrical signal intensity of the electrical signal sensor of the present disclosure significantly increases. Therefore, the electrical signal sensor can sense the tested sample in a trace amount, and the lifetime of using the electrical signal sensor is prolonged. For example, the above-mentioned Embodiment 1 is an example shown in FIG. 6. FIG. 6 is a diagram of the relative current density in the electrical signal sensor changing with time. In FIG. 6, curve C4 corresponds to Embodiment 1. In FIG. 6, the electrical signal sensor of Embodiment 1 senses 180 mg/dl of glucose per day for 18 consecutive days. As shown in FIG. 6, the relative current density maintains at the initial value of 1.0 (current density ratio having no unit), and the change is less than 20%. After sensing for at least 18 days, the electrical signal sensor still has no significant current signal degradation.

In summary, the electrical signal sensing composition, the electrical signal sensor including the electrical signal sensing composition, and the method of forming the electrical signal sensor in the present disclosure have a good electronic transfer in the electrical signal sensing composition to improve the performance of the electrical signal sensor, such as increased electrical signal, smaller detection limit, increases detection range, better detection sensitivity, etc.

The present disclosure is described in considerable detail in some embodiments. However, other embodiments may be feasible. Therefore, the description of the embodiments in the present disclosure should not limit the scope and spirit of the attached claims.

For one skilled in the art, the present disclosure may be modified and changed without deviating from the scope and spirit of the present disclosure. As long as the modifications and changes are within the scope and spirit of the attached claims, these modifications and changes are covered by the present disclosure.

What is claimed is:

1. A method of forming an electrical signal sensor, comprising:
dissolving an oxidoreductase and an amphiphilic molecule into a solvent to form a solution, wherein the amphiphilic molecule comprises alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof;
coating the solution on an electrode layer; and
drying the solution.

2. The method of claim 1, wherein the amphiphilic molecule comprises sodium n-octyl sulfate, sodium n-decyl sulfate, sodium dodecyl sulfate, sodium n-tetradecyl sulfate, or combinations thereof.

3. The method of claim 1, wherein the oxidoreductase comprises glucose oxidase, glucose dehydrogenase, pyruvate oxidase, catalase, xanthine oxidase, acetylcholinesterase, lactate oxidase, urate oxidase, pyrroloquinoline quinine glucose dehydrogenase-A, pyrroloquinoline quinine glucose dehydrogenase-B, NAD(P)-dependent glutamate dehydrogenase, FAD-dependent glutamate dehydrogenase, uricase, cholesterol oxidase, sulfurase, or combinations thereof.

4. The method of claim 1, wherein the oxidoreductase comprises an aggregated particle, and a particle size of the aggregated particle is from 10 nm to 5000 nm.

5. The method of claim 4, wherein the particle size of the aggregated particle is from 50 nm to 1000 nm.

6. The method of claim 1, wherein a weight ratio of the oxidoreductase to the amphiphilic molecule is from 1:0.1 to 1:50.

7. The method of claim 6, wherein the weight ratio of the oxidoreductase to the amphiphilic molecule is from 1:0.1 to 1:10.

8. The method of claim 1, wherein the electrode layer comprises gold, silver, platinum, aluminum, iridium, titanium, steel, stainless steel, gold alloy, platinum alloy, aluminum alloy, iridium alloy, titanium alloy, or combinations thereof.

9. The method of claim 1, wherein dissolving the oxidoreductase and the amphiphilic molecule into the solvent to form the solution is performed at a temperature from 20° C. to 30° C.

10. The method of claim 1, wherein coating the solution on the electrode layer is performed by dropping the solution onto an upper surface of the electrode layer.

11. A method of forming an electrical signal sensor, comprising:
disposing an electron transfer layer on an electrode layer;
coating a solution on the electron transfer layer, wherein the solution comprises an oxidoreductase, an amphiphilic molecule, and a solvent, wherein the amphiphilic molecule comprises alkyl sulfuric acid, alkyl sulfate, alkyl sulfonic acid, alkyl sulfonate, alkyl ammonium, alkyl ammonium salt, alkyl phosphoric acid, alkyl phosphate, alkyl carboxylic acid, alkyl carboxylate, alkylboronic acid, alkylborate, or combinations thereof; and
drying the solution.

12. The method of claim 11, wherein the amphiphilic molecule comprises sodium n-octyl sulfate, sodium n-decyl sulfate, sodium dodecyl sulfate, sodium n-tetradecyl sulfate, or combinations thereof.

13. The method of claim 11, wherein the oxidoreductase comprises glucose oxidase, glucose dehydrogenase, pyruvate oxidase, catalase, xanthine oxidase, acetylcholinesterase, lactate oxidase, urate oxidase, pyrroloquinoline quinine glucose dehydrogenase-A,, pyrroloquinoline quinine glucose dehydrogenase-B, NAD(P)-dependent glutamate dehydrogenase, FAD-dependent glutamate dehydrogenase, uricase, cholesterol oxidase, sulfurase, or combinations thereof.

14. The method of claim 11, wherein the electron transfer layer comprises a conductive carbon material, a conductive polymer, or a combination thereof.

15. The method of claim 14, wherein the conductive carbon material comprises graphite, graphene, single-wall carbon nanotube, multi-wall carbon nanotube, or combinations thereof, and the conductive polymer comprises polypyrrole, polyaniline, polythiophene, poly(3,4-ethylenedioxythiophene), poly-p-styrene, polyacetylene, polyphenylacetylene, polyphenylene sulfide, polybenzene, polythiazole, or combinations thereof.
